(19) United States
(12) Patent Application Publication
(10) Pub. No.: US 2026/0248739 A1
(43) Pub. Date: Aug. 27, 2026

(54) LIPID FORMULATIONS

(71) Applicant: SISAF LTD, Guildford (GB)

(72) Inventors: Roghieh Suzanne SAFFIE-SIEBERT, Guildford (GB); Flavia SUTERA, Guildford (GB); Nasrollah TORABI-POUR, Guildford (GB); Ashkan DEHSORKHI, Guildford (GB); Negeen KARGAR, Guildford (GB)

(73) Assignee: SISAF LTD, Guildford (GB)

(21) Appl. No.: 18/992,535

(22) PCT Filed: Jul. 21, 2023
(86) PCT No.: PCT/GB2023/051930

§ 371 Date: Jan. 8, 2025

(30) Foreign Application Priority Data
Jul. 22, 2022 (GB) 2210794.0

(51) Int. Cl. A61K 9/51; A61K 31/7105; A61K 31/711; A61K 38/00; A61K 39/00; A61K 39/12; A61K 47/24; A61K 48/00

(52) U.S. Cl. CPC A61K 9/5192; A61K 9/5115; A61K 9/5123; A61K 9/5146; A61K 31/7105; A61K 31/711; A61K 38/00; A61K 39/0011; A61K 39/12; A61K 47/24; A61K 48/005; A61K 2039/53; A61K 2039/55555

(57) ABSTRACT

A method of manufacturing hybrid lipid particles suitable for delivery of a fragile active ingredient such as a nucleic acid comprising the steps of making hybrid lipid particles and then “dusting” their surface with particles of an inorganic material. Also related hybrid lipid particles, uses and products, a key feature being that the particles of inorganic material are present on and in the lipid particle rather than being encapsulated by it and that the active ingredient also does not need to be encapsulated.

### FIELD OF THE INVENTION

The invention relates to improved lipid particles for delivery of nucleic acids. More specifically, the invention relates to the use of hydrolysable elemental silicon for improving the stability of lipid particles for the delivery of nucleic acids.

### BACKGROUND OF THE INVENTION

Lipid particles, including lipid nanoparticles (LNPs) show much promise as vectors for the delivery of therapeutic nucleic acid and in particular for the delivery of therapeutic RNA.

Many compounds which have potential for useful application, for example as active pharmaceutical ingredients, are prone to degradation. Nucleic acid, such as RNA is especially prone to degradation during formulation and storage. Various pharmaceutical compositions that seek to limit the extent of RNA degradation have been proposed. One way of limiting RNA degradation is to seek to encapsulate the RNA in a protective organic envelope of lipid. For example, EP3677567A1 discloses lipid particles which have mRNA molecules encapsulated within the particles.

The present invention is based, in part, on the appreciation and avoidance of some of the technical challenges of encapsulating nucleic acid within a lipid particle. Chief among the challenges is the necessity to form lipid particles at an elevated temperature. The precise temperature required depends on the lipids used, but typically temperatures of about 60° C. need to be employed. Such elevated temperatures cause significant degradation of RNA and other fragile active ingredients. This can be countered, to an extent, by use of modified active ingredients, for example modified RNA. It is notable that U.S. Pat. No. 9,504,651 B2 discloses a method of forming lipid particles “around” an mRNA wherein at least 70% of the mRNA is encapsulated. Of the unencapsulated 30%, it appears that much is degraded.

There are also related challenges in keeping lipid particles “stable”. Improving “stability” includes both countering the tendency of smaller lipid particles to coalesce into larger particles and also countering their tendency to lose the charges of the charged lipids which are part of the lipid particle. Much work has been carried out in the field of producing novel lipids with advantageous properties, and in developing formulations of multiple lipids which have advantageous properties in their ability to form stable particles. One advantage of the present invention lies in improvements in the stability of lipid particles. Another advantage of the present invention lies in the provision of methods to improve particle stability which are less dependent on the use of specific novel lipids which may be subject to technical challenges, supply constraints and intellectual property restrictions on their use. By using methods and products of the invention, it is possible to form sufficiently-stable lipid particles with a wider range of lipids including lipids which are of lower cost and more readily available then some of the specialist lipids which may need to be used in prior art methods and products in order to obtain lipid particles having sufficient performance. Conversely, the invention may also be used with advantageous prior art “high performance” specialised lipids to achieve even more superior performance.

Lipid in lipid particles can be configured in various ways to form liposome particles or to form micellar particles. Liposome particles essentially consist of bilayers of lipid molecules. Micellar particles generally have lipid in a non-bilayer configuration. Particles of the present invention are referred to herein as “hybrid lipid particles” meaning that they may have regions of micellar lipid configuration and regions of liposomal lipid configuration. Depending on pH hybrid lipid particles may adopt a liposomal configuration, for example due to deprotonation at neutral pH. According to preferred embodiments, less than 20% (by weight) of the total lipid is present in a liposomal (bilayer) configuration, meaning that between 80% and 100% (by weight) of the total lipid is present in a non-liposomal, for example, micellar, configuration. According to certain embodiments, the hybrid lipid particles are micellar lipid particles.

The present invention is based on an appreciation that in organic particles and in particular particles of silicon (and particles containing hydrolysable silicon) can be used to stabilise hybrid lipid particles, to inhibit their coalescence, to assist cationic lipids and/or ionisable lipids in retaining their charge and to promote the ability of the hybrid lipid particles to protect nucleic acid, not by encapsulation but by stabilising the nucleic acid on the hybrid lipid particle surface. Such an arrangement advantageously allows the hybrid lipid particles to be prepared in the absence of nucleic acid and the nucleic acid added to the particle only after they have been fully formed and any processes involving elevated temperature have been completed.

The background art discloses a variety of lipid particles for use in various methods. For example, CN106177892 discloses hyaluronic acid decorated lipid particles having a mesoporous core of silica. These particles are disclosed as being complexed with a drug by mixing the silica core with the drug and then adding the lipid in a subsequent step. CN114146188 discloses formulation of CoQ10 as a medically useful compound. It discloses the manufacture of a carrier from silica to silicon, lip encapsulations and then the addition of CoQ10. US2022/183989, WO2020/193999 and Baran-Rachwalska et al (2020) J. Cont. Rel: 326: 192-202 all disclose various particles comprising silicon or silica particles which have been coated with lipid.

It can be noted that these particles are essentially configured as lipid surround a silica/silicon core. Obviously, such a configuration will typically result in a particle which is larger overall than the core, but only slightly.

### SUMMARY OF THE INVENTION

According to a first aspect of the invention, there is provided a method of manufacturing hybrid lipid particles suitable for delivery of a fragile active ingredient (for example, for delivery of nucleic acid) comprising the steps of:
A. blending one or more lipid components, then
B. forming the lipid blend into a plurality of hybrid particles having a mean diameter of between 50 nm and 400 nm, and contacting the plurality of lipid particles with particles of inorganic material having a mean diameter of between 10 nm and 100 nm;
wherein the hybrid lipid particles have a mean diameter of at least 150% of the mean diameter of the particles of inorganic material.

According to a second aspect of the invention there is provided, a plurality of hybrid lipid particles comprising a blend of one or more lipid components, wherein the hybrid lipid particles have a mean diameter of between 50 and 400 nm and a coating of particles of inorganic material having a mean diameter of between 10 and 100 nm, said plurality of hybrid lipid particles having the ability to bind molecules of an active ingredient (such as nucleic acid molecules) to their surface and optionally comprising molecules of the active ingredient, (such as nucleic acid molecules) bound to their surface, wherein the hybrid lipid particles have a mean diameter of at least 150% of the mean diameter of the particles of inorganic material.

In further aspects the invention provides:

A pharmaceutical composition comprising a plurality of hybrid lipid particles according to the first aspect of the invention and a pharmaceutically-acceptable carrier.

A method of providing a vaccination to an individual in need thereof, comprising administering a pharmaceutical formulation of the invention, wherein the active ingredient is a nucleic acid molecule which is an mRNA molecule encoding an antigen, or part of an antigen, of a pathogenic organism or virus, or wherein the active ingredient is a protein or peptide antigen of a pathogenic organism or virus.

A method of providing a vaccination to an individual in need thereof, comprising administering a pharmaceutical formulation of the invention, wherein the active ingredient is a nucleic acid molecule that is an mRNA molecule encoding a tumour antigen, or part of a tumour antigen, or wherein the active ingredient is protein or peptide tumour antigen or portion thereof.

A method of treating a disease associated with the expression of a gene in an individual, by administering to said individual a pharmaceutical formulation of the invention, wherein the nucleic acid molecule is an siRNA capable of silencing the gene.

A plurality of hybrid lipid particles of the invention, or a pharmaceutical composition of the invention for use as a medicament.

A plurality of hybrid lipid particles or a pharmaceutical composition of the invention for use as a vaccine.

Use of a plurality of hybrid lipid particles of the invention, or a pharmaceutical composition of the invention in the manufacture of a medicament.

Use of a plurality of hybrid lipid particles of the invention, or a pharmaceutical composition of the invention in the manufacture of a vaccine.

### BRIEF DESCRIPTION OF THE DRAWINGS

FIG. 1 is a diagrammatic illustration of a proposed mechanism of action of the invention.

FIG. 2 shows data relating to RNA stability when the RNA is incorporated into a hybrid lipid particle of the invention.

FIG. 3 shows a schematic for manufacture of certain products of the invention.

FIG. 4 is a TEM photograph of a hybrid lipid particle of the invention.

As used herein hybrid lipid particles refers to particles comprising at least some lipid (as defined herein) in which the lipid molecules (at least 80% by weight in preferred embodiment) present to arrange themselves into a hybrid structure which is not a lipid bilayer (i.e., a structure having at least some micellar character). Hybrid lipid particles are distinct from liposomes which comprise a lipid bilayer, but the term “hybrid lipid particles” as used herein admits at least some liposome-type structures within the particle.

The term “hybrid” is intended to describe lipid particles containing at least some lipid in a configuration other than lipid bilayers. The amount of lipid in such a configuration may be from 80 to 0% (preferably less than 20%) by percentages and weight of total lipid.

### DETAILED DESCRIPTION OF THE INVENTION

According to a first aspect of the invention, there is provided a method of manufacturing hybrid lipid particles suitable for delivery of an active ingredient such as a fragile active ingredient (for example, for nucleic acid delivery) comprising the steps of:
A. blending one or more lipid components, then
B. forming the lipid blend into a plurality of lipid particles having a mean diameter of between 40 nm and 400 nm and contacting the plurality of of organic materials such as lipid particles with particles of inorganic material having a mean diameter of between 10 nm and 100 nm;
wherein the hybrid lipid particles have a mean diameter of at least 150% of the mean diameter of the particles of inorganic material.

According to preferred embodiments of the first aspect of the invention, there is provided a method of manufacturing hybrid lipid particles suitable for delivery of an active ingredient such as a fragile active ingredient (for example for, nucleic acid delivery) comprising the steps of:
A. blending one or more cationic lipid or ionisable lipid with one or more further lipid selected from neutral lipids and polar lipids, and optionally one or more additional lipid components, then
B. forming the lipid blend into a plurality of lipid particles having a mean diameter of between 40 nm and 400 nm, and contacting the plurality of lipid particles with particles containing hydrolysable silicon having a mean diameter of between 10 nm and 100 nm, then
C. contacting the hybrid lipid particles with a solution of the active ingredient (for example a solution of nucleic acid molecules) such that the molecules of the active ingredient (for example, nucleic acid molecules) electrostatically-bind to the cationic charge of the cationic lipid and ionisable lipid and thereby to the surface of the hybrid lipid particles.

According to a second aspect of the invention there is provided, a plurality of hybrid lipid particles comprising a blend of one or more lipid components, wherein the hybrid lipid particles have a mean diameter of between 50 and 400 nm and a coating of particles of inorganic material having a mean diameter of between 10 nm and 100 nm, such a plurality of hybrid lipid particles having the ability to bind molecules of an active ingredient such as nucleic acid molecules to their surface and optionally comprising molecules the active ingredient such as nucleic acid molecules bound to their surface, wherein the hybrid lipid particles have a mean diameter of at least 150% of the mean diameter of the particles of inorganic material.

Without wishing to be bound by theory, it is suggested that the hybrid lipid particles of the invention are held together by a combination of electrostatic, covalent and van der Waals forces. The organic part of the particle is thought to stabilize the inorganic part via the formation of resonance bands, for example carboxylic acid groups where the positive charge is delocalized between the bands. When an active ingredient such as a nucleic acid binds to this delocalized charge, it condenses. Condensation increases as the charges increase. The presence of the particles of inorganic material increases hydrogen banding and facilitates active ingredient binding and allows for better condensation of nucleic acid.

According to preferred embodiments of the second aspect of the invention there is provided, a plurality of hybrid lipid particles comprising a blend of one or more cationic lipids or ionisable lipids, with one or more polar lipids, and optionally one or more additional lipid components, wherein the hybrid lipid particles have a mean diameter of between 50 and 400 nm and preferably of between 80 nm and 400 nm and a coating particles containing hydrolysable silicon having a mean diameter of between 10 nm and 60 nm and preferably of between10 nm and 60 nm, such a plurality of hybrid lipid particles having the ability to bind nucleic acid molecules to their surface and optionally comprising molecules of an active ingredient such as nucleic acid molecules bound to their surface, wherein the hybrid lipid particles have a mean diameter of at least 150% of the mean diameter of the particles of inorganic material.

In further aspects the invention is as described above.

Lipids, in the present technical field, are generally understood to include fatty acids and fatty acid derivatives, glycerolipids, glycerophospholipids, sphingolipids, saccharolipids and polyketides.

As used in the present application, the term “lipid” also covers lipidated oligopeptides (a term used interchangeably herein with the term lipopeptide) wherein a short peptide sequence (such as a peptide sequence having 3 to 20 amino acid residues, such as 5 to 15 amino acid residues, especially 3, 4, or 5 amino acid residues, and most especially 5 amino acid residues) is conjugated to one or more fatty acid chains (especially a fatty acid chain having a 10 to 24 carbon chain length, preferably, a 12 to 18 carbon chain length; for example a 14, 15 or 16 carbon chain length; for instance, the peptide moiety may optionally be lipidated with a palmitoyl, cetyl or myristoyl moiety).

A lipidated oligopeptide may optionally be a lipdated tetrapeptide, lipidated pentapeptide or lipidated hexapeptide. Preferably, the amino acid residues include at least one amino acid residue (for example, 2 or 3 amino acid residues) that is cationic at a pH of 7.4 (physiological pH), such as lysine or arginine. For example, the lipidated oligopeptide may include one or more (for example 2) lysine resides. Thus, a particular example is palmitoyl-pentapeptide-4 (CAS number 214047-00-4):

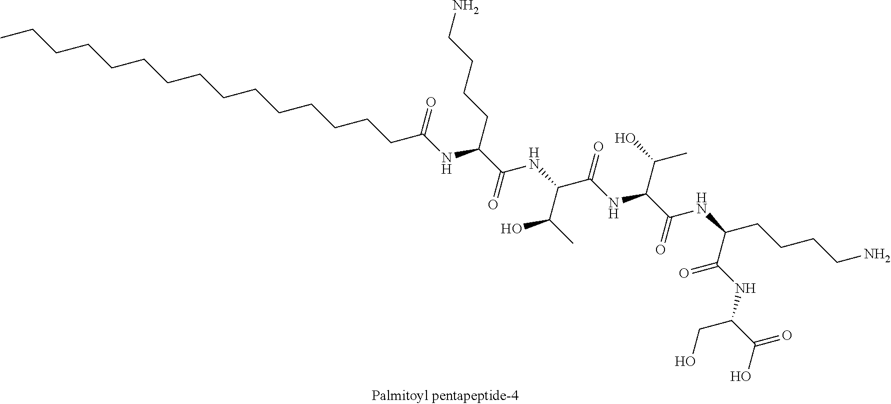 [SMILES: CCCCCCCCCCCCCCCC(=O)N[C@@H](CCCCN)C(=O)N[C@H](C(=O)N[C@H](C(=O)N[C@@H](CCCCN)C(=O)N[C@@H](CO)C(=O)O)[C@@H](C)O)[C@@H](C)O]

Thus, in certain preferred embodiments according to all aspects of the invention, the one or more lipids is or comprises one or more lipidated oligopeptides, particularly those having one or more amino acid residues that is or are cationic at a pH of 7.4 (physiological pH; examples include lysine and arginine). Depending on the characteristics of the peptide component of the lipopeptide, the lipopeptide may be a cationic lipid, an ionisable lipid, a neutral lipid, or a polar lipid. According to various embodiments of the invention, the requirement for one of more of cationic lipid, ionisable lipid, neutral lipid or polar lipid may be met by a suitable lipopeptide. Alternatively, or additionally, one or more additional lipid components of products an method of the invention may be provided by a suitable lipopeptide.

The lipidated oligopeptide may be used in combination with one or more phospholipids, such as DOPE or DPPC. It is thought that the alkyl chain of a lipopeptide may advantageously be assimilated in a phospholipid bilayer, while the surface of the bilayer is decorated with the peptide moiety. It is thought that in this way, the peptide may provide for tissue and/or cell targeting; and, for example where the peptide bears a cationic charge at physiological pH, may stabilise negatively charged active ingredients, such as nucleic acid, such as mRNA.

However, it has been found that DOTAP is not always necessary for the present formulations, therefore, in certain preferred embodiments, the one or more lipids is, are or comprise(s) one or more of a phospholipid (such as DPPC and/or DOPE) and a lipidated oligopeptide having one or more amino acid residues that is or are cationic at a pH of 7.4 (physiological pH; examples include lysine and arginine). Optionally also present are one or more sugars (particularly trehalose) and/or one or more amino acids (particularly glycine).

Alternatively, in other preferred embodiments, the one or more lipids are or comprise one or more of a phospholipid (such as DPPC and/or DOPE), and are formulated with one or more coenzymes (for example, NAD); one or more flavanols (for example, quercetin) and/or one or more amino acids (for example, glycine, tyrosine). Optionally also present are one or more sugars (particularly trehalose) and/or one or more amino acids (particularly glycine).

According to all aspects of the invention the blend of one or more cationic lipid or, ionisable lipid with one or more neutral lipid or polar lipids comprises at least one cationic lipid or ionisable lipid. Preferably the total cationic lipid or ionisable lipid (as a molar ratio) is between 20 and 70% of the total lipid, for example between 30 and 60%, or between 40 and 60%.

According to certain embodiments the cationic lipid or ionisable lipid is a cationic lipid. In additional to a suitable lipopeptide, the cationic lipid may be selected from the group consisting of DOTAP (dioleoyl-3-trimethylammonium propane, 18:1 TAP); DODAC (dimethyldioctadecylam-moniumchloride): SA (stearylamine, octadecylamine) and DOTMA (9-(trimethyl[2,3-(dioleyloxy) propyl]ammonium chloride) and mixtures of any thereof. Mixtures comprising DOTAP are especially preferred. According to certain embodiments at least half or all of the cationic lipid is DOTAP. According to certain embodiments at least half or all of the cationic lipid is a cationic lipopetide.

According to other embodiments the cationic lipid or ionisable lipid is an ionisable lipid. The ionisable lipid may be selected from the group consisting of [(4-hydroxybutyl)azanediyl]di(hexane-6,1-diyl) bis(2-hexyldecanoate, heptadecan-9-yl 8-{(2-hydroxyethyl)[6-oxo-6-(undecyloxy)hexyl]amino}octanoate, 7-[(2-hydroxyethyl) [8-(nonyloxy)-8-oxooctyl]amino]heptyl 2-octyldecanoate, (6Z,9Z,28Z,31Z)-heptatriaconta-6,9,28,31-tetraen-19-yl 4-(dimethylamino)butanoate, and DODMA, and mixtures of any thereof.

According to certain embodiments, the cationic lipid or ionisable lipid component may be a mixture of one or more cationic lipid (for example one or more of the cationic lipids listed above) and one or more ionisable lipid (for example one or more of the ionisable lipids listed above).

The lipid blend may optionally further include one or more neutral or polar lipid. The neutral phospholipid DOPE (dioleoyl phophatrolylethanolamine), PC (phosphatrdyl chlorine) and lecithin (a mixture predominated by PC) are all examples of non-cationic phospholipids which may be used as the neutral or polar lipid in accordance with the invention.

According to certain preferred embodiments the lipid blend is wholly or predominantly a cationic lipid and a phospholipid. For example, the lipid blend may consist of DOTAP and DOPE in approximately equal amounts. In another example, the lipid blend may consist of a cationic lipopeptide.

The lipid blend may optionally and additionally further comprise as an additional lipid component a conjugated lipid such as a PEG-lylated lipid, PEI, Brij58 (surfactant) and/or a steroid/sterol component such as cholesterol. According to certain preferred embodiments, the lipid blend does not contain a material amount of either a conjugated lipid nor a material amount of a steroid/sterol. In certain embodiments, the additional lipid may comprise or consist of an additional lipopeptide. Steroid/sterols in accordance with the invention may optionally be of animal origin and/or may optionally be of non-animal origin (for example of plant origin, or microbial origin).

The lipid bend may optionally or additionally further comprise mitoxantrone (MTO) or a derivative thereof such as dipalmitoyl MTO or monopalmitoyl MTO.

### Particle Sizes

According to all embodiments, the mean diameter of the hybrid lipid particles of the invention is significantly larger than the mean diameter of the particles of inorganic material. According to preferred embodiments the mean diameter of the hybrid lipid particles is of between 80 nm and 400 nm and the mean diameter of the particles of inorganic material is between 10 nm and 60 nm. In those embodiments (and other embodiments wherein one or both of the mean diameters is more narrowly defined), the feature of the hybrid lipid particles having a mean diameter of at least 150% of the mean diameter of the particles of inorganic materials is optional.

### Manufacture of Hybrid Lipid Particles

Hybrid lipid particles of the second aspect of the invention may be manufactured by any suitable method. They may optionally be manufactured by a method of the first aspect of the invention. For example, a lipid blend as described above may be prepared. In certain embodiments it may be prepared by blending lipids in a suitable solvent. Optionally the lipid blend in the solvent may be dried by evaporation of solvent using any suitable method. A rotary evaporator may optionally be used to produce a thin layer of anhydrous lipid blend, or any other methods known in the field of lipid particles production such as microfludiser or tangential flow filtration (TFF). Hybrid lipid particles may be produced by hydrating the anhydrous lipid blend, for example, by contact it with an aqueous solution. These steps are carried out prior to the introduction of the active ingredient such as a nucleic acid and may therefore safely and optionally involve elevated temperature and chemical conditions which are not conducive to stability of the active ingredient (for example elevated temperatures and chemical conditions not conducive to RNA stability and which would tend to cause degradation and loss of RNA molecules).

Following formation of hybrid lipid particles, methods of, and relating to, the invention subsequently provide stabilization of the newly formed hybrid lipid particles by treating them with particles of inorganic material, preferably hydrolysable silicon particles. Such a treatment may be described as a “coating”. By “coating it is to be understood that the particles of inorganic material (preferably hydrolysable silicon particles) will be predominantly present at or towards the surface of the hybrid lipid particle. They may be partially embedded within the lipid particle but will be at least partially accessible on the surface of the hybrid lipid particle. Preferably, the particles of inorganic material (hydrolysable silicon particles) are of a significantly smaller diameter than the hybrid lipid particles. For example, the particles of inorganic material (hydrolysable silicon particles) may be at least half or at least a third or at least a quarter, or at least a sixth, eighth or tenth of the mean diameter of the hybrid lipid particles. The particles of inorganic material (hydrolysable silicon particles) may be mixed with the hybrid lipid particles at a wider range of ratios. For example, the particles of inorganic material (hydrolysable silicon particles) may be present at about 0.1× to 10× the amount of lipid.

### Filtration

Following the addition of particles of inorganic material. (for example, hydrolysable silicon particles) to the lipid particles, the resultant particles comprising hybrid lipid with an inorganic particle (preferably hydrolysable silicon particle) coating may optionally be filtered. According to the first aspect of the invention a filtration step may be interposed between steps B and C. Preferably this is carried out using tangential flow filtration although any suitable filtration process could be used. One purpose of filtration is to increase the size uniformity of the hybrid lipid particles by means of filtration through a membrane having a size cut-off at the desired particle size (for example 100 nm).

After filtration, the total proportion of the weight of the hybrid lipid particle made up from the inorganic particle (hydrolysable silicon) may be somewhat reduced.

### Note on Particle Sizes

This specification describes particle sizes as mean diameters. Particle diameters may be measured by any suitable method including electron microscopy and size exclusion methods. Preferably, the particles have a distribution of diameters around the mean diameter that is such that 80% of the particles have a diameter within 25%±the mean diameter. This is especially the case following filtration which is known to increase mono-dispersity.

### Hydrolysable Silicon

“Hydrolysable silicon” as used herein encompasses pure elemental silicon. However complete purity is not required. Conversely the invention is not intended to cover pure silica (including sand, quartz, silica gel). A key requirement is that the material is hydrolysable, that is to say, that it will tend to breakdown under physiological conditions to soluble products such as orthosilicic acid (OSA). According to certain embodiments the definition “particle containing hydrolysable silicon” is met if at least half of the mass of the material is hydrolysed to soluble products within a month of injection into a subject (for example following intramuscular or subcutaneous injection).

Hydrolysable silicon according to the invention is preferably mesoporous. This is to say, it contains pores having diameters between 2 to 50 nm in diameter.

Particles containing hydrolysable silicon may be purchased commercially or may be produced by any suitable method. For example, bulk silicon may be reduced by milling or electro-etching. Silicon particles or desired sized may be obtained by any suitable method, for example by us of an air-classifier.

### Doped Silicon

It is preferred that the hydrolysable silicon for use in all aspects of the invention comprises (or consists of) doped silicon. Silicon may be n-doped or p-doped. Most preferably, the silicon is p-doped. Most preferably, the silicon is p-doped with boron.

Most preferably, the doping is heavy which is understood to mean one or more dopant atom is added per 10,000 silicon atoms or even “super doped” for example boron doped of 1020 atoms/cm³ depends on the need to generate different zeta charge inside the crystal matrix &/or on the for surface of hybrid system.

The semiconductor industry provides a ready source of appropriately doped silicon as well as technology for doping.

### Active Ingredients

In preferred embodiments, active ingredients of the invention are active pharmaceutical ingredients. Multiple active ingredients may be present in a single formulation in accordance with the invention, but it is more usual for each embodiment of the invention to concern a single API. According to certain preferred embodiments the active ingredient is an API which is a nucleic acid. Most preferable it is an RNA, such as a mRNA, an siRNA, a haRNA or a saRNA. In preferred embodiments the active ingredient is negatively charged (i.e., has a net negative charge at physiological pH). Active ingredients (APIs) according to the invention my preferably be fragile active ingredients. Fragile active ingredients may be understood to be active ingredients having poor stability. For example, active ingredients have poor stability at 20° C. and pH 7.4, such as those having a half-life of less than 6 days, or less than 1 day.

### Bindings of Nucleic Acid to Silicon Lipid Particles

Hybrid lipid particles of the invention bind active ingredient such as nucleic acid to their surface in contrast to prior art lipid particles which seek to encapsulate the active ingredient such as nucleic acid within the lipid structure. Due to the electrostatic forces and the concentration gradient of the resulting organic-inorganic hybrid system, active ingredients tend to be incorporated into the matrix and surface of silicon. Since nucleic acid could itself act as a charge stabilizer, ionized drugs, even if hydrophobic or hydrophilic, will tend to distribute themselves along the surface of mesoporous silicon. Regions of the mesoporous Silicon functionalized with nucleic acid will tend to associate hydrophilic ionized drugs due to the resonance of electrons generated on the surface-polar molecules interact through dipole-dipole intermolecular forces and hydrogen bonds.

Binding of active ingredient such as nucleic acid to the surface is possible because of electrostatic attraction between a negatively charged active ingredient such as a negatively-charged nucleic acid and the positive charge proximal to the head of the cationic lipids constituting the hybrid lipid particles &/or within silicon matrix.

Whilst it may be possible to achieve the kind of electrostatic co-ordination with prior art lipid particles containing cationic lipid but no inorganic material (preferably, hydrolysable silicon), long term attraction of active ingredient such as nucleic acid is difficult to maintain with prior art particles because cationic lipids have a tendency to lose charge in storage. This is a process known as lipid ageing. The presence of inorganic material (preferably, hydrolysable silicon) in accordance with the present invention allows the cationic lipid or charged lipid including ionisable lipids to maintain its charge by stabilizing that charge through electrostatic co-ordination. To create a stabilize charge and complex the inventors further refers the importance of the degree of binding of nucleic acid on the silicon particle surface will depend also on the surface contact angle. The Surface contact angle and consequently the dissolution behavior depends on the “ratio” of hydroxide and oxide-terminated fragments on silicon particle surface. So, a careful functionalization of inorganic silicon particle surface, as the resulting combination of both size of Si porous and chemistry surface is required for controlling dissolution rate, stabilizing charge and the final kinetic of release.

### Nucleic Acid for Use in the Invention

The invention is suitable for use with any active ingredient. It is preferred that the active ingredient is nucleic acid. The invention is especially useful for use with RNA because RNA, in the absence of the protection provided by the invention, is especially prone to degradation. Therefore, according to certain preferred embodiments of all aspects of the invention, the nucleic acid is RNA. RNA may optionally be siRNA. It may optionally be mRNA. For example, it may be mRNA encoding a vaccine antigen. RNA may optionally be chemically-modified or sequence-modiefied to increase its stability and prevent its degradation. According to certain embodiments of the invention the RNA is chemically-modified to increase its stability or to prevent its degradation. However, in certain preferred embodiments the RNA is not chemically-modified because such treatment has been found to be unnecessary.

According to certain embodiments of all aspects of the invention, the nucleic acid is DNA. According to other preferred embodiments, the nucleic acid is RNA. It may be siRNA or mRNA. It may be of any suitable length but typically may be between 10 and 30 nucleotides long for siRNA or 200 to 2000 nucleotides long for mRNA. It may be double or single stranded or, especially in the case of siRNA it may be chemically single stranded but with one or more regions of base pairing (and with optional unpaired overhangs). It may optionally be modified chemically (for example by use of N1-methylpseudouridine substitution) or have its sequence modified (for example by UTR-shortening). Preferably, the nucleic acid (i.e., RNA) may be unmodified (especially not chemically modified) as this may be unnecessary to provide stability.

### Configuration of Particle

Hybrid lipid particles of the invention, in all its aspects, preferably have the following configuration in addition to the hybrid lipid structure. The hybrid lipid particles have particles of inorganic material (preferably, hydrolysable silicon particles) both integrated in the lipid bilayer and also partially or wholly exposed on the particle surface so as to be available to interact with nucleic acid. Preferably, at least 50% of total particles of inorganic material (preferably, hydrolysable silicon particles) are accessible at the surface of the lipid particle and are not fully encapsulated within the micellar structure. When present the active ingredient such as nucleic acid is predominantly located electrostatically bound to the surface of the particle. For example, over 90% of the total active ingredient such as nucleic acid present will be bound to the surface of the particles and less than 10% will be encapsulated within the lipid structure before fully condensed and created a complex. In some embodiments zero or virtually zero (for example less than 0.5%) of the total active ingredient such as nucleic acid present will be encapsulated within the structure.

### Additional Components

It has been found that the stability of the hybrid lipid particle can be increased further in the presence of one or more amino acids and/or one or more non-reducing disaccharides. Therefore, methods of the invention may optionally be carried out in the presence of one or more amino acid and/or one or more non-reducing disaccharide. The hybrid lipid particles of all aspects of the invention may further comprise a non-reducing disaccharide and/or an amino acid. A preferred non-reducing disaccharide is trehalose. A preferred amino acid is glycerine. In certain preferred embodiments the use of both glycine and trehalose is preferred. These components may be able to electrostatically-coordinate with the particles of inorganic material (preferably hydrolysable silicon) and increase the overall stability of the system.

### Stability of Particle

The hybrid lipid particles of the invention show enhanced dimensional-stability compared to corresponding particles without the particles of inorganic material (preferably, hydrolysable silicon) particles according to the invention. This enhanced dimensional stability manifests in a resistance of the particles to coalescence into larger particles. According to certain embodiments, the rate of coalescence at 5° C. is at least half that of equivalent corresponding particles (of identical composition but for the absence of particles of inorganic material (preferably, hydrolysable silicon particles) according to the invention). According to certain embodiments, at least 90% of particles have not coalesced and have maintained their original size after 3 months storage in aqueous solution at physiological pH at 5° C.

### Stability of Charge

The surface charge of hybrid lipid particles may be estimated using the parameter of zeta potential (eletrokinetic potential). As a rule of thumb, a suspension of particles having a low zeta potential (O to ±5 mV) is unstable and rapidly coalesces. Values of ±30 mV to ±40 mV correspond to reasonable stability and values ±40 mV to ±60 mV good stability, values above ±60 mV correspond to excellent stability.

According to certain embodiments, the hybrid lipid particles of the invention have values of >±40 mV, more preferably >±45 mV, >±50 mV, or >±60 mV. Preferably the zeta potential is increased by at least ±10 mV by the presence of hydrolysable silicon (that is to say the zeta potential is at least ±10 mV greater than +10 mV than that of lipid particles which are identical to those of the invention but for the absence of particles of inorganic material (preferably, hydrolysable silicon particles) according to the invention).

The presence of inorganic material (preferably silicon) also inhibits loss of positive charge of the cationic lipids. This is known as lipid ageing and preferably is slowed (at 5° C.) by at least a factor of 2, 4, 8 or 16.

### Stability of Active Ingredient Such as Nucleic Acid

The hybrid lipid particles of the invention act to protect active ingredient such as nucleic acid (in particular RNA) complexed electrostatically to the surface of the hybrid lipid particles. The invention allows therapeutic formulations such as vaccines to be stored with greater ease. For example, to be stored at 5° C. or room temperature as opposed sub-zero temperatures. It also increases stability and reduces active ingredient such as nucleic acid degradation during freeze-drying, rehydration, transport and storage. According to certain embodiments of the invention the half-life of an active ingredient such as an mRNA is extended by a factor of at least 100, at least 1000 or at least 10000 compared with a corresponding active ingredient such as mRNA not complexed with a hybrid lipid particle of the invention. According to certain embodiments, the half-life of an active ingredient such as an mRNA is extended by a factor of at least 10, at least 100 or at least 1000 compared to a corresponding active ingredient such as an mRNA complexed with an equivalent hybrid lipid particle lacking the inorganic particle (preferably, hydrolysable silicon) component according to the invention. Half-lives may be measured at pH7 at 5° C. in aqueous physiological solution (for example, in a phosphate buffered saline solution).

Formulation into Pharmaceutical Products

The invention further contemplates the use of hybrid lipid particles of the invention to formulate pharmaceutical products which also fall within the scope of the invention. Such pharmaceutical products include injectables (such as injectable vaccines), topical creams, capsules, tablets, and ointments. They also include pharmaceutical precursors or products for example dehydrated (lyophilized) and concentrated products which must be diluted and/or rehydrated prior to use.

### Methods of, and Products Relating to, Treatment

Products of the invention may be used in methods of treatment or may be products for use in methods of treatment. Methods of the invention may further comprise subsequent steps constituting methods of treatment.

Methods of treatment include treatment or prevention of disease. In some embodiments, methods of treatment may comprise down-regulation of gene expression by siRNA. In other embodiments, methods of treatment may comprise vaccination, for example, vaccination against a cancer or vaccination against an infectious disease by delivery of an mRNA encoding an antigen (or fragment thereof) or the causative agent of the infectious disease (for example the spike protein of SARS-CoV-2). Alternatively, a peptide or protein antigen corresponding to a tumour antigen (or fragment thereof) may be administered as a vaccine.

Optionally, in certain preferred embodiments of all aspects of the invention, the open reading frame of the mRNA encodes a tumour-specific antigen. As used herein, the term tumour-specific antigen may refer to an antigen that arises, in one or more malignant cancer cells, from non-synonymous somatic mutation (leading to a neoantigen) or viral-integrated mutation (leading to an oncoviral antigen). Tumor-specific antigens may thus refer to antigens that are completely absent from (not expressed by) non-cancerous (healthy, normal) cells.

Optionally, the open reading frame of the mRNA encodes a tumour-associated antigen. As used herein, the term tumour-associated antigen may refer to an antigen that is over-expressed in a malignant cancer cell, compared to a non-cancerous (healthy, normal) cell, for example due to genetic amplification or post-translational modifications. The term tumour-associated antigen may encompass overexpressed antigens (which term may refer to proteins that are moderately expressed in non-cancerous (healthy, normal) cells, but expressed abundantly in malignant cancer cells); differentiation antigens (which term may refer to proteins that are selectively expressed by the cell lineage from which the malignant cells evolved, an example being prostate-specific antigen); and cancer-germline antigens (which term may refer to antigens that are normally limited to reproductive tissues, but which are aberrantly expressed in a malignant cancer cell; for example, melanoma antigen family A3 (MAGE-A3); New York Esophageal Squamous Cell Carcinoma-1 Antigen (NY-ESO-1); and Preferentially Expressed Antigen in Melanoma (PRAME))

When the open reading frame of the mRNA encodes a cancer-associated antigen or a cancer-specific antigen, the nucleic acid products of the invention may be suitable for use in a prophylactic or therapeutic vaccine composition.

Optionally, the open reading frame of the mRNA encodes an allergen (including but not limited to one or more nut allegens; which in turn include but are not limited to: one or more seed storage proteins, such as vicilins, legumins, albumins; one or more plant defense related proteins; and one or more profilins).

In some embodiments, methods of treatment may comprise delivery of a biologically active compound.

### Proposed Mechanism of Action.

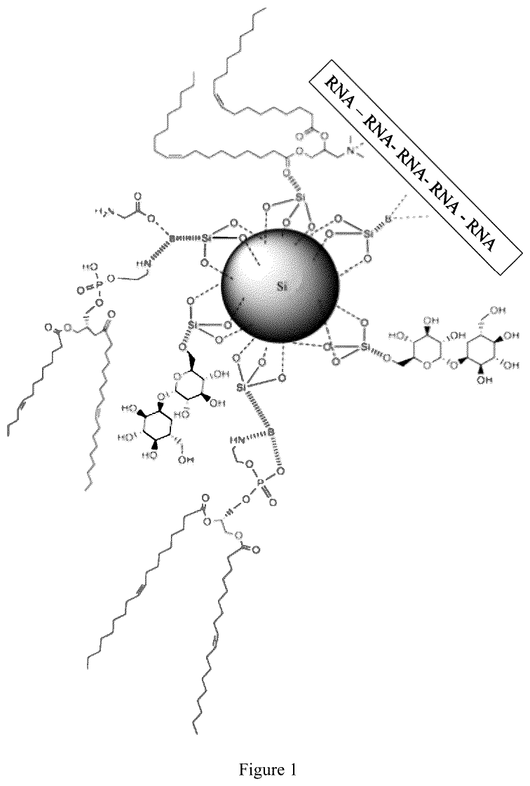

Without wishing to be bound by theory and using formulations comprising hydrolysable silicon to illustrate the broader principle, attention is drawn to FIG. 1 which illustrates a proposed mechanism of action of the invention in its various aspects. The centre of the drawing shows diagrammatically, a particle containing hydrolysable silicon. Selected Si atoms are highlighted as are certain O atoms. It should be noted that even nominally “Pure” elemental silicon is unlikely to have surface Si atoms bound to other chemical moieties such as O and —OH. The diagram illustrates possible modes of electrostatic coordination between glycine and trehalose and silicon and boron atoms of the hydrolysable silicon particle. The diagram also illustrates electrostatic coordination between atoms of the hydrolysable silicon particle and phospholipid and between atoms of the hydrolysable silicon particle and DOTAP as an example of a cationic lipid. The diagram illustrates a length of RNA (as an example of an active agent) which of course has negative charges long its length and illustrates a plausible mechanism by which those negative charges and thereby the RNA molecule itself is stabilised by coordination with atoms of the hydrolysable silicon particle and with charges on the lipid (which is itself stabilised by coordination with atoms of the hydrolysable silicon particle.

Various aspects of the invention are illustrated in the following non-limiting examples.

### EXAMPLES

### Example 1. Exemplary Method of Manufacturing Products of the Invention

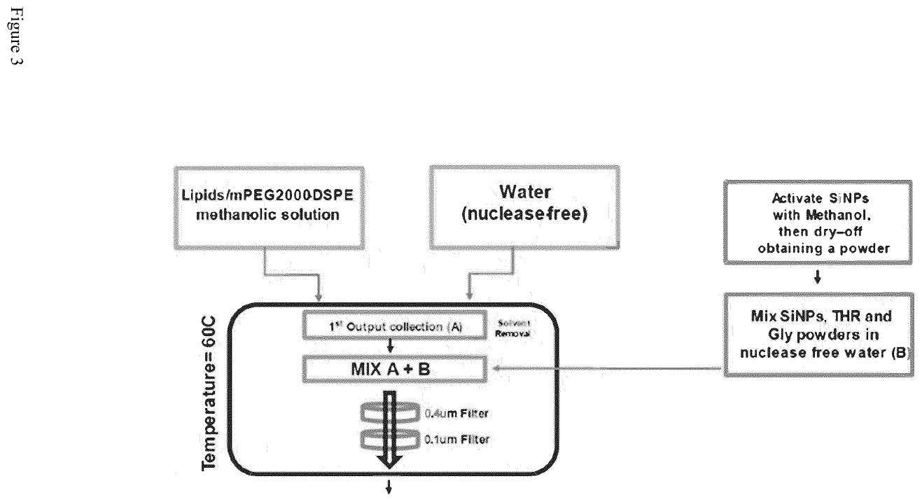

The manufacturing process of products of the invention is shown schemtically in FIG. 3 starts with dissolving the selected lipids DOTAP, DOPE and mPEG2000-DSPE in methanol. At the same time, porous silicon nanoparticles are activated by exposure to methanol. Subsequently, the solvent is evaporated in a slow evaporation process to generate the activated SiNPs. This activation step is aimed at rendering the SiNPs amenable to dispersion in water. The activated SiNPs are then dispersed in (nuclease-free) water in the presence of Trehalose (THR) and Glycine (GLY). An appropriate volume of lipids is successively transferred to a round bottom flask and the methanol evaporated by rotary evaporation. As a result of this evaporation step, a lipid film is generated on the wall of the flask. The suspension containing the dispersed Silicon, THR and GLY is then added to the flask containing the lipid thin film for lipid rehydration. Successively, passage through over 0.4 μm and 0.1 μm extrusion membranes at 60° C. is performed. Extruded samples are then stored under refrigerated conditions. The amounts of each component is shown below:

| | | | | | | Activated | | | |
|---|---|---|---|---|---|---|---|---|---|
| Lipids | | | | | | SiNPs AE | | | Nuclease- |
| DOTAP | | DOPE | | mPEG2000-DSPE | | (≤100 nm) | Glycine | Trehalose | free water |
| 7.25 | mg | 7.30 | mg | 1.45 | mg | 1 mg | 0.5 mg | 1 mg | Up to 10 mL |

| | | | | | | | |
|---|---|---|---|---|---|---|---|
| 1.45 | mL | 1.46 | mL | 0.29 | mL | 1 mL | Up to 10 mL |

### Example 2. Demonstration that the Presence of Silicon Prevents Particle Coalescence and Promotes the of Retention of Surface Charge

Particles made in accordance with the invention (and also comparator particles) were kept at 5° C. and periodically assesses for size (diameter in nm), size dispersity (poly dispersity index-PDI) and Zeta potential (ZP, which is indicative of surface charge). Sample SIS0012 was made with undoped silicon, SIS0013 was made with boron-doped silicon and NoSiNP was made without silicon and the results are shown below. Characterisation for both size and surface charge used a Malvern Zetasizer Advanced Pro series instrument. Samples were prepared using 20 μl of particles of the invention with 980 μl nuclease free water to make a total of 1000 μl which was placed inside a disposable cuvette. Measurements were done by performing 3 scans to reduce signal to noise interference. The average of the 3 scans were calculated as final size measurement along with the poly disperse index (PDI).

Zeta potential measurements were done by mixing 200 μl of particles of the invention with 800 μl of nuclease free water (for SIS0012 with silicon) or 1 mM KCl (for NoSiNP to ensure good conductivity). 5 scans were averaged to determine the zeta potential given in the table below.

| | Week 0 | | | 4 weeks | | | 12 weeks | | | 24 weeks | | |
|---|---|---|---|---|---|---|---|---|---|---|---|---|
| | Size | | ZP | Size | | ZP | Size | | ZP | Size | | ZP |
| Sample | (nm) | PDI | (mV) | (nm) | PDI | (mV) | (nm) | PDI | (mV) | (nm) | PDI | (mV) |
| No SiNP | 113.5 | 0.08 | 69 | 112.4 | 0.10 | 52 | 134.6 | 0.191 | 27.96 | 151.8 | 0.07 | 30.7 |
| SIS0012 | 94.28 | 0.234 | 52.71 | 96.5 | 0.200 | 54.15 | 87.14 | 0.201 | 53.19 | 90.58 | 0.09 | 61.52 |
| SIS0013 | 102.3 | 0.230 | 55.12 | 99.89 | 0.211 | 54.19 | 78.23 | 0.245 | 50.12 | 81.94 | 0.06 | 62.92 |

It can be seen that the size of particles formulated without silicon tends to increase with time as particles coalesce and that this tendency is not seen when silicon is present. Similarly, a loss of surface charge is seen in particles formulated without silicon which is absent in those in which silicon is present.

### Example 3. Demonstration of Ability to Stabilise RNA

mRNA (Dasher GFP mRNA (Lot Number: 88103F, Aldevron) stock solution was prepared by dissolving the mRNA powder in nuclease-free water to a concentration of 0.5 μg/μL. The mRNA was complexed with hybrid lipid particles of the invention (prepared as in Example 1, samples coded SIS0012 used undoped silicon, samples coded SIS0013 used boron doped silicon) were prepared by mixing 50 μL (25 μg) of mRNA solution with 200 μL of hybrid lipid particle suspension (hybrid lipid particle/RNA weight ratio 12:1). The mixtures were incubated at room temperature for 40 min to allow for complete complexation and then either used in the liquid form or freeze-dried. The freeze dried complexes were then embedded into sodium hyaluronate hydrogels (the freeze-dried complexes were directly mixed with the hydrogel, whilst the liquid complexes were analysed as such). The final preparations were prepared as 30 μL aliquots to minimize the risk of cross-contamination during storage and analysis, and were stored either at room temperature or at 40° C.

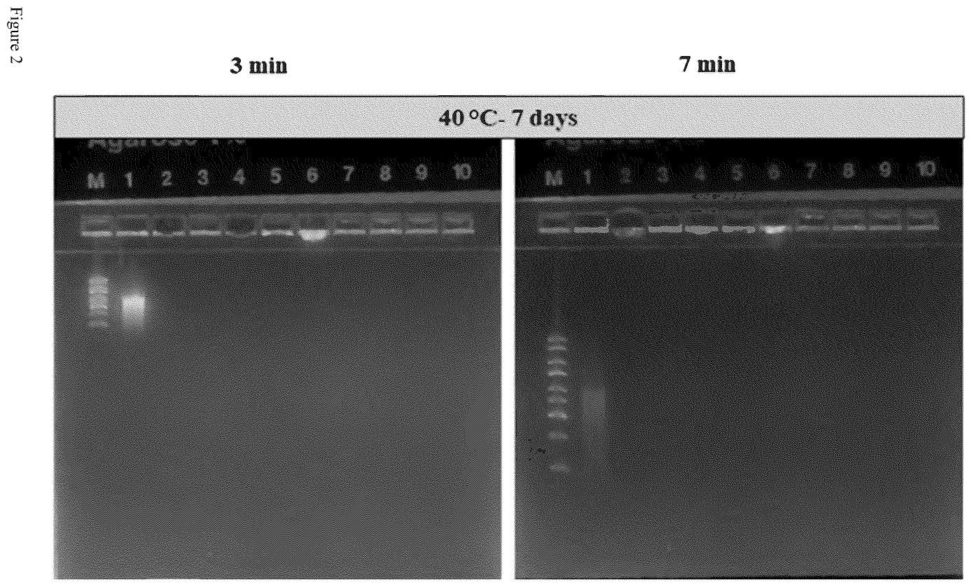

After storage for 7 days, the samples were subjected to gel electrophoresis. Naked mRNA stored in similar conditions was used as control in all cases and a DNA ladder was used as a size guide. The samples were loaded onto the E-Gel™ agarose gel (1%) in the E-Gel™ Power Snap Electrophoresis Device. The gel was transilluminated and imaged after 3 min and 7 min of electrophoresis using the E-Gel™ Power Snap Electrophoresis camera. The total amount of mRNA loaded onto the gel for different samples are provided in the table below:

| Gel lane | | Amount of |
|---|---|---|
| (FIG. 2) | Sample | mRNA (μg/well) |
| 2 | mRNA-SIS0012 (Freeze-dried in hydrogel) | 2 |
| 3 | mRNA-SIS0012(liquid) | 2 |
| 4 | mRNA-SIS0013 (Freeze-dried in hydrogel) | 2 |
| 5 | mRNA-SIS0013(liquid) | 2 |
| 6 | mRNA-SIS0012(Freeze-dried in hydrogel) | 2 |
| 7 | mRNA-SIS0012(liquid) | 2 |
| 1 | Naked mRNA | 1 |

As can be seen from FIG. 2, degraded mRNA is highly mobile, and the degraded fragments produce a smear on the gel. This is seen with naked mRNA. The mRNA complexed with the hybrid lipid particles of the invention fails to degrade and retains its original length meaning that it is much less electrophoretically mobile and (at 3 and 7 minutes) has yet to migrate appreciably from the loading well of the gel.

### Example 4—Use of Protein (Alkaline Phosphatase) in a Formulation of the Invention

Alkaline phosphatase is an enzyme that exists in various forms, catalyses the degradation of various proteins, and may be found in all tissues in the human body. It is mostly concentrated in the bones, kidneys, liver, intestines, and placenta. It contributes, inter alia, to: protection of the intestinal tract against bacteria; digestive function; degradation of fats and vitamin B; and bone formation. Alkaline phosphatase exhibits a loss of activity at low pH and at high temperature. Alkaline phosphatase activity can be monitored by measuring changes in the concentration, for which UV-Vis absorbance is a proxy, of one or more of its substrates or products in an in vitro assay. For example, the concentration of the substrate 4-nitrophenyl phosphatase (PNPP) the structure of which is set out below, may be monitored to track the following reaction:

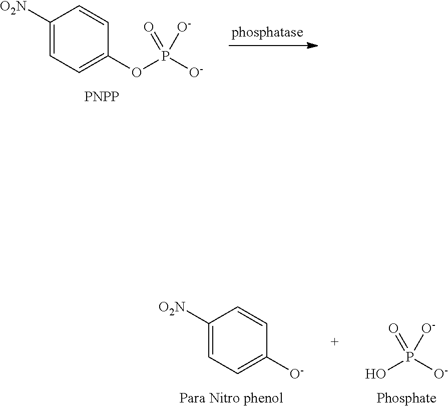

### Materials, Methods and Results

Alkaline phosphatase, isolated from bovine intestine and supplied as a recombinant enzyme of 56 kD, expressed in the yeast, Pichia Pastoris, was obtained from Sigma Aldrich/Merck (The Old Brickyard, New Rd, Gillingham, Dorset, SP8 4XT). An aqueous stock solution of alkaline phosphatase (ALP) was prepared at a concentration of 1 U/ml. 1 U (μmol/min) is defined as the amount of ALP that catalyzes the conversion of one micromole of PNPP per minute at 37° C. and pH 7.4.

A 20 mM solution of 4-nitrophenyl phosphatase (PNPP) was prepared using Tris Buffer at pH 7.4 (100 mM/L).

ALP solutions were prepared at the following concentrations:
0.1, 0.5, 1, 5, 10, 50, and 100 mU/ml
from the 1 U/ml stock solution, in 15 ml test tubes.

These were then mixed, in Eppendorf tubes, with the prepared 20 mM solution of PNPP Tris buffer.

The tubes were incubated in a water bath at 37° C. for 30 minutes, following which, UV-Vis absorbance measurements were made at 405 nm as shown in FIG. 22.

ALP was loaded onto hybrid lipid particles of the invention as follows:

1. 3 sets of 8 Eppendorf tubes were prepared:
A 8 Eppendorf tubes were prepared, each containing 50 μL of ALP (50mU/ml) and 500 μL of hybrid lipid particles (comprising undoped Si). After adding these components to the tubes, they were mixed, vortexed and refrigerated overnight.
B 8 Eppendorf tubes were prepared, each containing 50 μL of ALP (50mU/ml) and 500 μL of hybrid lipid particles (comprising boron doped Si). After adding these components to the tubes, they were mixed, vortexed and refrigerated overnight.
C 8 Eppendorf tubes were prepared, each containing 50 μL of ALP (50mU/ml) and 500 μL of Tris buffer. After adding these components to the tubes, they were mixed, vortexed and refrigerated overnight.

2. Following their preparation, all Eppendorf tubes were placed in a water bath at 50° C. Each of the eight tubes, in each of the three sets of tubes A to C, was removed from the water bath after 1, 2, 5, 10, 20, 40 or 60 minutes.

3. 300 μL of PNPP was then added to all Eppendorf tubes in all sets A to C. The tubes were mixed and vortexed. They were then placed in a water bath at 37° C. for 30 minutes, during which time dephosphorylation of the PNPP occurred.

4. Following this, UV-Vis analysis may be carried out on all samples in all sets A to C (at 405 nm) in order to assess the ability to stabilise the protein active ingredient (ALP).

### Imaging of Particles

In order to better understand the configuration of the hybrid lipid particles of the invention, TEM images were obtained.

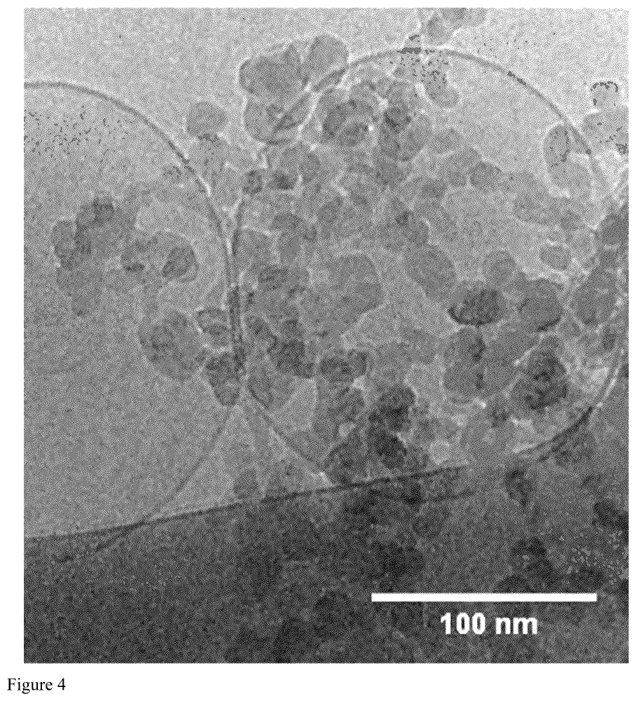

FIG. 4 is an example of one of these images and shows a hybrid lipid particle prior to loading with an active ingredient. The large spheres having diameters of about 120 nm are lipid particles and the smaller particles are of silicon. As can be seen, the silicon is irregular and in substantially smaller particles. It can also be seen that whilst some silicon particles may be embedded in the lipid particle, there are also silicon particles are associated with the surface of the lipid particles. Note that this image was obtained following filtration, meaning that any silicon present is sufficiently well-bound to the lipid particle to survive the filtration process.

1. A method of manufacturing hybrid lipid particles suitable for delivery of a fragile active ingredient comprising the steps of:
A. blending one or more lipid components, then
B. forming the lipid blend into a plurality of lipid particles having a mean diameter of between 50 nm and 400 nm, and contacting the plurality of hybrid lipid particles with particles of inorganic material having a mean diameter of between 10 nm and 100 nm;
wherein the hybrid lipid particles have a mean diameter of at least 150% of the mean diameter of the particles of inorganic material.

2. A method according to claim 1, wherein the particles of inorganic material are less than one quarter of the diameter of the hybrid lipid particles and wherein they are not encapsulated or incompletely encapsulated by lipid.

3. A method according to claim 1, wherein step A comprises blending one or more cationic lipids or ionisable lipids with one or more further lipids selected from neutral lipids and polar lipids.

4. A method according to claim 1, wherein the method comprises as an additional step after step B, a step of
C. contacting the hybrid lipid particles with a solution of active ingredient having a net negative charge at pH 7.4 such that the active ingredient binds electrostatically to the cationic charge of the cationic lipid and/or ionisable lipid and thereby to the surface of the hybrid lipid particles.

5. A method according to claim 1, wherein the particles of inorganic material are particles containing hydrolysable silicon.

6. (canceled)

7. A method according to claim 3, wherein the one or more cationic lipids or ionisable lipids is a cationic lipid selected from the group consisting of DOTAP, DODAC, SA or DOTMA or mixtures thereof.

8. (canceled)

9. A method according to claim 3, wherein the one or more further lipids is a polar phospholipid selected from DOPE, PC and lecithin or mixtures thereof.

10. (canceled)

11. (canceled)

12. (canceled)

13. (canceled)

14. (canceled)

15. (canceled)

16. A method according to claim 1, wherein the active ingredient is DNA comprises nucleic acid molecules.

17. A method according to claim 16, wherein the active ingredient is DNA, mRNA, siRNA, saRNA or hairpin RNA.

18. A method according to claim 17, wherein the mRNA encodes an antigen, or part of an antigen, of a pathogen or cancer, or wherein the siRNA inhibits the expression of a gene associated with a disease in subject.

19. (canceled)

20. (canceled)

21. (canceled)

22. A method according to claim 1, wherein more than 90% of the active ingredient present is bound to the outer surface of the hybrid lipid particle and less than 10% of the total active ingredient present is encapsulated within the hybrid lipid particle.

23. A method according to claim 1, comprising the further step, subsequent to steps A and B, of freeze-drying the hybrid lipid particles.

24. A method according to claim 1, comprising a further step D subsequent to steps A and B, wherein step D comprises formulating the hybrid lipid particles into a pharmaceutical composition.

25. (canceled)

26. A method according to claim 24, wherein one or more of steps A, B or D are carried out in the presence of one or more amino acid molecules and/or one or more molecules of a non-reducing disaccharide.

27. (canceled)

28. (canceled)

29. (canceled)

30. A plurality of hybrid lipid particles comprising a blend of one or more cationic lipids or ionisable lipids, with one or more polar lipids, wherein the hybrid lipid particles have a mean diameter of between 50 and 400 nm and a coating of particles containing hydrolysable silicon having a mean diameter of between 10 and 100 nm, said plurality of hybrid lipid particles having the ability to bind nucleic acid molecules to their surface, wherein the hybrid lipid particles have a mean diameter of at least 150% of the mean diameter of the particles of inorganic material.

31. A plurality of hybrid lipid particles according to claim 30, further comprising an active pharmaceutical ingredient bound to their surface.

32. A plurality of hybrid lipid particles according to claim 31, wherein the active ingredient comprises nucleic acid molecules.

33. A plurality of hybrid lipid particles according to claim 32, where the nucleic acid molecules are DNA, mRNA, siRNA, saRNA or hairpin RNA.

34. A plurality of hybrid lipid particles according to claim 30, wherein the one or more cationic lipids or ionisable lipids is a cationic lipid selected from the group consisting of DOTAP, DODAC, SA or DOTMA or mixtures thereof.

35. (canceled)

36. (canceled)

37. (canceled)

38. A method of providing a vaccination to an individual in need thereof, comprising administering a pharmaceutical formulation comprising a plurality of hybrid lipid particles according to claim 30 and a pharmaceutically acceptable carrier, wherein the active ingredient is an mRNA molecule encoding an antigen, or part of an antigen, of a pathogenic organism or virus, a peptide or protein antigen or part of an antigen, a tumour antigen, part of a tumour antigen, or a peptide or protein tumour antigen or part thereof.

39. (canceled)

40. (canceled)

41. (canceled)

42. (canceled)

43. (canceled)

44. (canceled)

45. A method according to claim 4, comprising the further step, subsequent to steps A to C, of freeze-drying the hybrid lipid particles.

46. A method according to claim 4, comprising a further step D subsequent to steps A to C, wherein step D comprises formulating the hybrid lipid particles into a pharmaceutical composition.

47. A method according to claim 46, wherein one or more of steps A to D are carried out in the presence of one or more amino acid molecules and/or one or more molecules of a non-reducing disaccharide.

48. A method of treating a disease associated with the expression of a gene in an individual, comprising administering a pharmaceutical formulation comprising a plurality of hybrid lipid particles according to claim 30 and a pharmaceutically acceptable carrier, wherein the active ingredient is an siRNA capable of silencing said gene; wherein the active ingredient is an mRNA molecule encoding an antigen, or part of an antigen, of a pathogenic organism or virus, a peptide or protein antigen or part of an antigen, a tumour antigen, part of a tumour antigen, or a peptide or protein tumour antigen or part thereof.
